(19) United States
(12) Patent Application Publication
(10) Pub. No.: US 2016/0000600 A1
(43) Pub. Date: Jan. 7, 2016

(54) Eyeball Cooling Module, Eyeball Cooling Apparatus Comprising Same, Eyeball Cooling Eye Patch Comprising Same, Eyeball Cooling Glasses Comprising Same and Eyeball Cooling Goggle Comprising Same

(71) Applicant: YONSEI UNIVERSITY WONJU INDUSTRY ACADEMIC CO-OPERATION FOUNDATION, Wonju-si Gangwon-do (KR)

(72) Inventor: Yong-heum Lee, Wonju-si (KR)

(73) Assignee: Yonsei University Wonju Industry-Academic Cooperation Foundation, Wonju-si (KR)

(21) Appl. No.: 14/767,773

(22) PCT Filed: May 9, 2013
(86) PCT No.: PCT/KR2013/004089

§ 371 Date: Aug. 13, 2015

(30) Foreign Application Priority Data
Feb. 27, 2013 (KR) 10-2013-0020843

(51) Int. Cl. A61F 7/00

(52) U.S. Cl. CPC A61F 7/00; A61F 7/007; A61F 2007/0004; A61F 2007/0075

(57) ABSTRACT

An eyeball cooling module includes a cooling device and a heat dissipation unit. The cooling device generates a cooling stimulation to human eyeballs by absorbing heat from a first side in which the human eyeballs are disposed and dissipating heat to a second side opposed to the first side. The heat dissipation unit is disposed at the second side of the cooling device, and dissipates heat from the second side of the cooling device externally. Accordingly, cooling stimulation to eyeballs of a human may be generated to human eyeballs, fatigue of eyes and eyeballs may be relieved at home and eyeball fever may be reduced at hospitals.

### THE ART TO WHICH THE INVENTIVE CONCEPT

Example embodiments generally relate to an eyeball cooling module, an eyeball cooling apparatus, an eyeball cooling eye-patch, eyeball cooling glasses and an eyeball cooling goggle having the eyeball cooling module, and more particularly to an eyeball cooling module for eyeball health care, an eyeball cooling apparatus, an eyeball cooling eye-patch, eyeball cooling glasses and an eyeball cooling goggle having the eyeball cooling module.

### BACKGROUND OF THE INVENTIVE CONCEPT

Recently, many humans experience an eyeball fever case due to increased use of mobile phone and computers. In addition, some humans experience an eyeball fever case due to eyeball disease and eyeball surgery.

However, the eyeball fever cannot be quickly and easily relieved. Therefore, there is need for a technique to relieve the eyeball fever quickly and easily.

### CONTENT OF THE INVENTIVE CONCEPT

### Technical Object of the Inventive Concept

Some example embodiments provide an eyeball cooling module capable of relieving the eyeball fever quickly and easily.

Some example embodiments provide an eyeball cooling apparatus including the eyeball cooling module.

Some example embodiments provide an eyeball cooling eyepatch including the eyeball cooling module.

Some example embodiments provide an eyeball cooling eyeglasses including the eyeball cooling module.

Some example embodiments provide an eyeball cooling goggle including the eyeball cooling module.

### Means for Achieving the Technical Object

According to some example embodiments, an eyeball cooling module includes a cooling device and a heat dissipation unit. The cooling device generates a cooling stimulation to human eyeballs by absorbing heat from a first side in which the human eyeballs are disposed and dissipating heat to a second side opposed to the first side. The heat dissipation unit is disposed at the second side of the cooling device, and dissipates heat from the second side of the cooling device externally.

In example embodiments, the cooling device may include a Peltier device. The Peltier device may include two metal plates spaced apart from each other. The Peltier device may absorb heat from the first side and may dissipate heat to the second side when a potential is induced between the two metal plates.

The Peltier device may include a heat absorption unit, a heat dissipation unit, a p-type device and an n-type device. The heat absorption unit may include a first metal plate and a second metal plate that are disposed at the first side and are spaced apart from each other. The potential may be induced between the first metal plate and the second metal plate. The heat dissipation unit may include a third metal plate disposed at the second side. The p-type device may be disposed between the first metal plate and the third metal plate. The n-type device may be disposed between the second metal plate and the third metal plate.

In example embodiments, the heat dissipation unit may include a heat dissipation pad, a heat dissipation plate and a heat dissipation pan which are disposed in consecutive order from the second side of the cooling device.

According to some example embodiments, an eyeball cooling apparatus includes an eyeball cooling module, a power supply unit and a cooling stimulation control unit. The eyeball cooling module includes a cooling device and a heat dissipation unit. The cooling device generates a cooling stimulation to human eyeballs by absorbing heat from a first side in which the human eyeballs are disposed and dissipating heat to a second side opposed to the first side. The heat dissipation unit is disposed at the second side of the cooling device, and dissipates heat from the second side of the cooling device externally. The power supply unit provides a power supply voltage to the eyeball cooling module. The cooling stimulation control unit outputs a cooling stimulation control signal that controls the cooling stimulation to the power supply unit.

In example embodiments, the power supply unit may control a level of the power supply voltage to control a strength of the cooling stimulation, in response to the cooling stimulation control signal.

In example embodiments, the power supply unit may control a supply time of the power supply voltage to control a time of the cooling stimulation, in response to the cooling stimulation control signal.

In example embodiments, the power supply unit may control a supply pattern of the power supply voltage to control a pattern of the cooling stimulation, in response to the cooling stimulation control signal.

In example embodiments, the cooling device may include a Peltier device that includes two metal plates spaced apart from each other. The Peltier device may absorb heat from the first side and may dissipate heat to the second side when a potential occurs between the two metal plates.

In example embodiments, the heat dissipation unit may include a heat dissipation pad, a heat dissipation plate and a heat dissipation pan which are disposed in consecutive order from the second side of the cooling device.

According to some example embodiments, an eyeball cooling eyepatch includes an eyeball cooling module, an acceptance unit and a flexible band. The eyeball cooling module includes a cooling device and a heat dissipation unit. The cooling device generates a cooling stimulation to human eyeballs by absorbing heat from a first side in which the human eyeballs are disposed and dissipating heat to a second side opposed to the first side. The acceptance unit is opposed to the human eyeballs, and accepts the eyeball cooling module. The flexible band is coupled from both ends of the acceptance unit.

According to some example embodiments, an eyeball cooling eyeglasses includes an eyeball cooling module, an acceptance unit and ear-holding units. The eyeball cooling module includes a cooling device and a heat dissipation unit. The cooling device generates a cooling stimulation to human eyeballs by absorbing heat from a first side in which the human eyeballs are disposed and dissipating heat to a second side opposed to the first side. The acceptance unit is opposed to the human eyeballs, and accepts the eyeball cooling module. The ear-holding units extend from both ends of the acceptance unit and have configurations to attach from human ears.

According to some example embodiments, an eyeball cooling goggle includes an eyeball cooling module, an acceptance unit and a flexible band. The eyeball cooling module includes a cooling device and a heat dissipation unit. The cooling device generates a cooling stimulation to human eyeballs by absorbing heat from a first side in which the human eyeballs are disposed and dissipating heat to a second side opposed to the first side. The acceptance unit is opposed to the human eyeballs, and accepts the eyeball cooling module. The acceptance unit includes heat absorption holes formed on a first surface opposed to the human eyeballs. The flexible band is coupled from both ends of the acceptance unit.

In example embodiments, the acceptance unit may further include first heat dissipation holes formed on a second surface opposed to the first surface.

The acceptance unit may further include second heat dissipation holes formed on a third surface perpendicular to the first surface and the second surface.

### Effects of the Inventive Concept

The eyeball cooling module, the eyeball cooling apparatus, the eyeball cooling eyepatch, the eyeball cooling eyeglasses and the eyeball cooling goggle according to example embodiments may generate cooling stimulation to eyeballs of a human. Therefore, the example embodiments may be used for relieving fatigue of eyes and eyeballs at home and may be used for reducing eyeball fever after operation on eyes and eyeballs at hospitals.

### BRIEF DESCRIPTION OF THE DRAWINGS

Example embodiments of the inventive concept will be more clearly understood from the following detailed description taken in conjunction with the accompanying drawings.

FIG. 1 is a block diagram illustrating an eyeball cooling apparatus according to example embodiments.

FIG. 2 is a perspective of an eyeball cooling apparatus of FIG. 1.

FIG. 3 is a block diagram illustrating the cooling device in FIGS. 1 and 2.

FIG. 4 is a perspective view illustrating an eyeball cooling eyepatch according to example embodiments.

FIG. 5 is a perspective view illustrating an eyeball cooling eyeglasses according to example embodiments.

FIG. 6A is a perspective view illustrating an eyeball cooling goggle according to example embodiments.

FIG. 6B is a front view illustrating an eyeball cooling goggle of FIG. 6A.

FIG. 6C is a rear view illustrating an eyeball cooling goggle of FIG. 6A.

FIG. 6D is a plain view illustrating an eyeball cooling goggle of FIG. 6A.

FIG. 6E is a side view illustrating an eyeball cooling goggle of FIG. 6A.

Explanation of reference numerals

| | |
|---|---|
| 100: an eyeball cooling | 110: an eyeball cooling module |
| apparatus | |
| 130: a power supply unit | 150: a cooling stimulation control unit |
| 210: cooling device | 220: a heat dissipation unit |
| 230: a heat dissipation pad | 250: a heat dissipation plate |
| 600: an eyeball cooling eyepatch | 700: an eyeball cooling eyeglasses |
| 800: an eyeball cooling goggle | |

### PARTICULAR CONTENTS FOR IMPLEMENTING THE INVENTIVE CONCEPT

Various example embodiments will be described more fully hereinafter with reference to the accompanying drawings, in which some example embodiments are shown. In describing principles of example embodiments, the detailed description of the well-known function and configuration may be omitted if such description may bur the inventive concept.

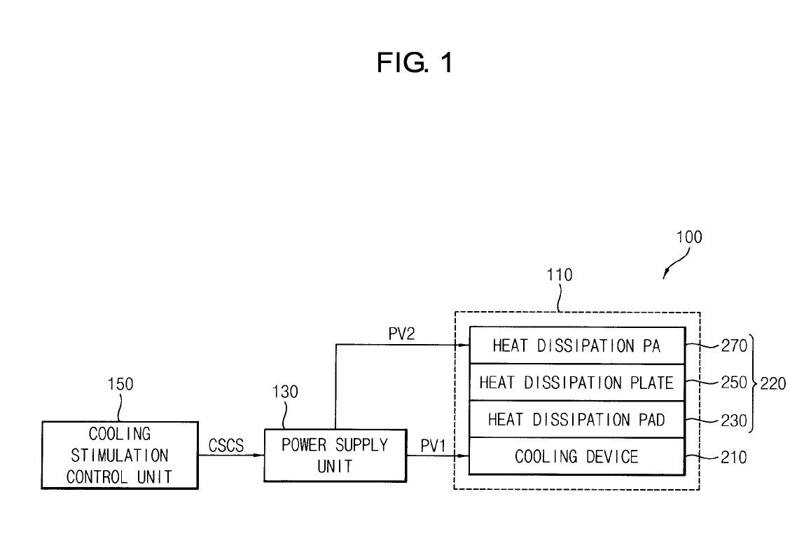

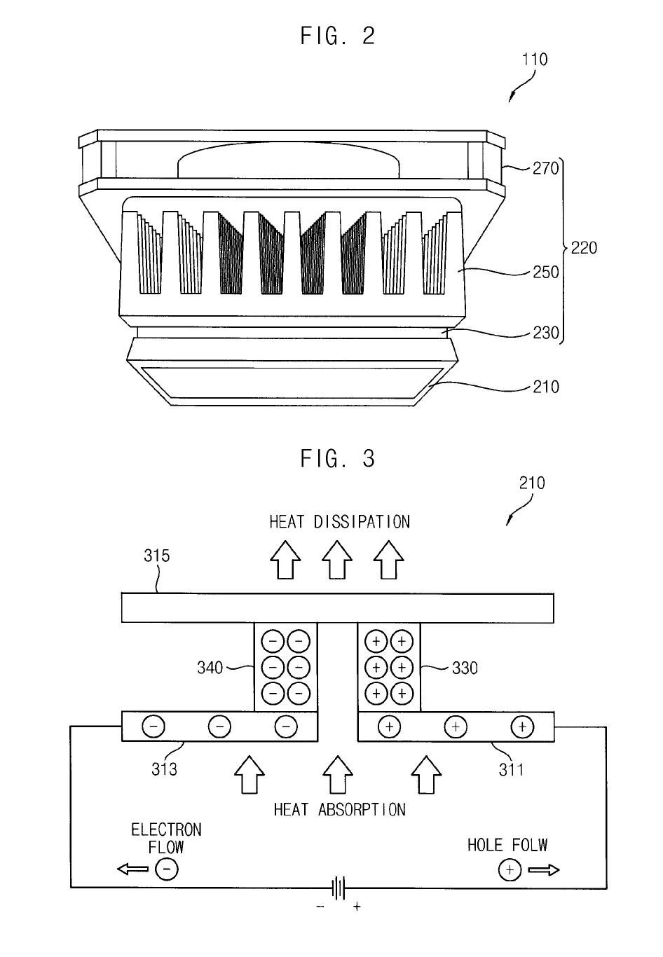

FIG. 1 is a block diagram illustrating an eyeball cooling apparatus according to example embodiments, and FIG. 2 is a perspective of an eyeball cooling apparatus of FIG. 1.

Referring to FIGS. 1 and 2, an eyeball cooling apparatus 100 includes an eyeball cooling module 110, a power supply unit 130, and a cooling stimulation control unit 150.

The eyeball cooling module 110 is disposed around eyes of a human and generates cooling stimulation to eyeballs of the human. That is, the eyeball cooling module 110 may reduce fever phenomenon occurring in the eyeballs of the human. The eyeball cooling module 110 includes a cooling device 210 and a heat dissipation unit 220.

The cooling device 110 generates a cooling stimulation to human eyeballs by absorbing heat from a first side in which the human eyeballs are disposed and dissipating heat to a second side opposed to the first side. For example, the cooling device 110 may include a Peltier device that includes two metal plates spaced apart from each other, and the Peltier device may absorb heat from the first side and dissipate heat to the second side when a potential occurs between the two metal plates.

FIG. 3 is a block diagram illustrating the cooling device in FIGS. 1 and 2.

Referring to FIG. 3, the cooling device 210 includes a first metal plate 311, a second metal plate 313, a third metal plate 315, a p-type device 330 and an n-type device 340. The first metal plate 311 and the second metal plate 313 are disposed at the first side and are spaced apart from each other. The first metal plate 311 and the second metal plate 313 may operate as a heat absorption that absorbs heat and the third metal plate 315 may operate as a heat dissipation unit that dissipates heat. The p-type device 330 is coupled between the first metal plate 311 and the third metal plate 315 and the n-type device 340 is coupled to the second metal plate 313 and the third metal plate 315.

When a power supply voltage having a positive polarity is applied to the first metal plate 311 and a power supply voltage having a negative polarity is applied to the second metal plate 313, the potential is induced between the first metal plate 311 and the second metal plate 313, holes move from the first metal plate 311 to the third metal plate 315 through the p-type device 330 and electrons move from the second metal plate 313 to the third metal plate 315 through the n-type device 340. The holes transfer heat, and thus the first metal plate 311 and the second metal plate 313 continuously become cooled and the third metal plate 315 continuously become heated. Therefore, the cooling device 210 generates the cooling stimulation to human eyeballs by absorbing heat from the first side dissipating heat to the second side.

Referring FIGS. 1 and 2 again, the heat dissipation unit 220 is disposed at the second side of the cooling device 210 and dissipates heat from the second side of the cooling device 210 externally. The heat dissipation unit220 includes a heat dissipation pad 230, a heat dissipation plate 250 and a heat dissipation pan 270. The heat dissipation pad 230, the heat dissipation plate 250 and the heat dissipation pan 270 are disposed in consecutive order from the second side of the cooling device 210.

The power supply unit 130 provides the cooling device 210 with a first power supply voltage PV1 for inducing the potential. The power supply unit 130 provides the heat dissipation unit fan 270 with a second power supply voltage PV2 for driving the heat dissipation fan 270. The power supply unit 130 may be electrically coupled to the eyeball cooling module 110 through a power cable. The power supply unit may be a battery. In this case, the power supply unit 130 may be coupled to the cooling device 210 and the heat dissipation fan 270 and the power supply unit 130 may be included in the eyeball cooling module 110.

The cooling stimulation control unit 150 controls the cooling stimulation generated by the cooling device 210. For example, the cooling stimulation control unit 150 outputs a cooling stimulation control signal CSCS that controls the cooling stimulation to the power supply unit 130.

In example embodiments, the power supply unit 130 controls the first power supply voltage PV1 output to the cooling device 210, in response to the cooling stimulation control signal CSCS. For example, the power supply unit 130 controls a level of the first power supply voltage PV1 to control a strength of the cooling stimulation, in response to the cooling stimulation control signal CSCS. For example, the power supply unit 130 controls a supply time of the first power supply voltage PV1 to control a time of the cooling stimulation, in response to the cooling stimulation control signal CSCS. For example, the power supply unit 130 controls a supply pattern of the first power supply voltage PV1 to control a pattern of the cooling stimulation, in response to the cooling stimulation control signal CSCS. The power supply unit 130 may perform pulse width modulation (PWM) on the first power supply voltage PV1 in response to the cooling stimulation control signal CSCS.

Accordingly, the eyeball fever may be quickly relieved by generating the cooling stimulation to the eyeballs of a human using the eyeball cooling apparatus 100.

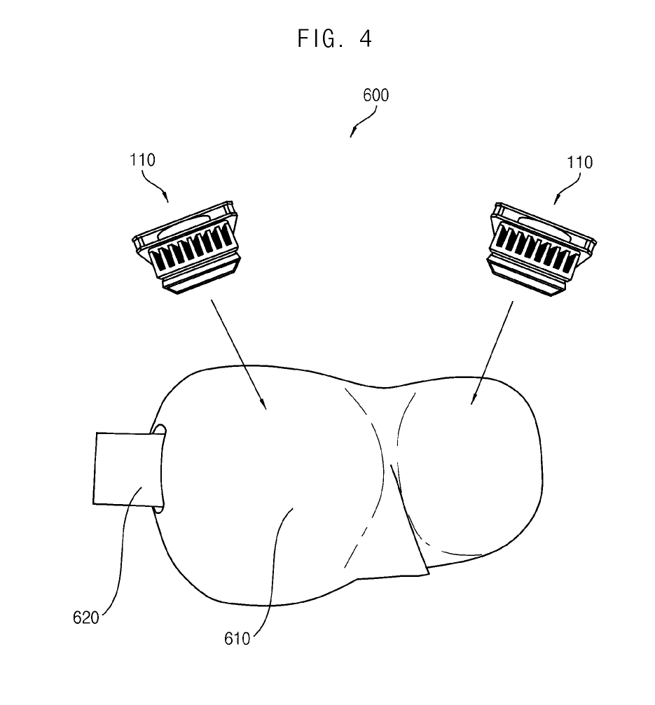

FIG. 4 is a perspective view illustrating an eyeball cooling eyepatch according to example embodiments.

Referring to FIG. 4, an eyeball cooling eyepatch 600 includes the eyeball cooling module 110, an acceptance unit 610 and a flexible band 620.

The eyeball cooling module 110 generates a cooling stimulation to eyeballs, is explained with reference to FIGS. 1 and 2, and thus, detailed description of the eyeball cooling module 110 will be omitted.

The acceptance unit 610 is opposed to a portion opposed to eyeballs of a human that wears the eyeball cooling eyepatch 600 and accepts the eyeball cooling module 110. The eyeball cooling module 110 is accepted in the acceptance unit 610 such that the heat absorption unit is opposed to the eyes of the human.

The flexible band 620 is coupled from both ends of the acceptance unit 610 to form a closed loop and prevents the eyeball cooling eyepatch 600 from rolling down.

Accordingly, the eyeball fever may be quickly and easily relieved by wearing the eyeball cooling eyepatch 600 that generates the cooling stimulation to the eyeballs of the human.

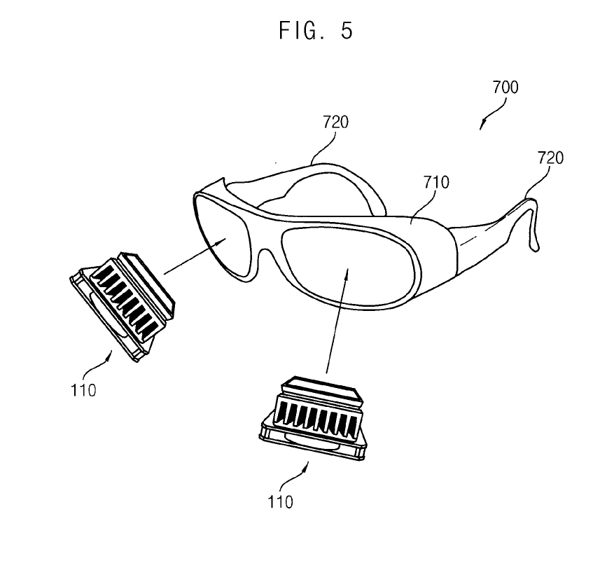

FIG. 5 is a perspective view illustrating an eyeball cooling eyeglasses according to example embodiments.

Referring to FIG. 5, an eyeball cooling eyeglasses 700 includes the eyeball cooling module 110, an acceptance unit 710 and ear-holding units 720.

The eyeball cooling module 110 generates a cooling stimulation to eyeballs, is explained with reference to FIGS. 1 and 2, and thus, detailed description of the eyeball cooling module 110 will be omitted.

The acceptance unit 710 is opposed to a portion opposed to eyeballs of a human that wears the eyeball cooling eyeglasses 700 and accepts the eyeball cooling module 110.

The eyeball cooling module 110 is accepted in the acceptance unit 710 such that the heat absorption unit is opposed to the eyes of the human.

The ear-holding units 720 extend from both ends of the acceptance unit 710 and have configurations to attach from human ears.

Accordingly, the eyeball fever may be quickly and easily relieved by wearing the eyeball cooling eyeglasses 700 that generates the cooling stimulation to the eyeballs of the human.

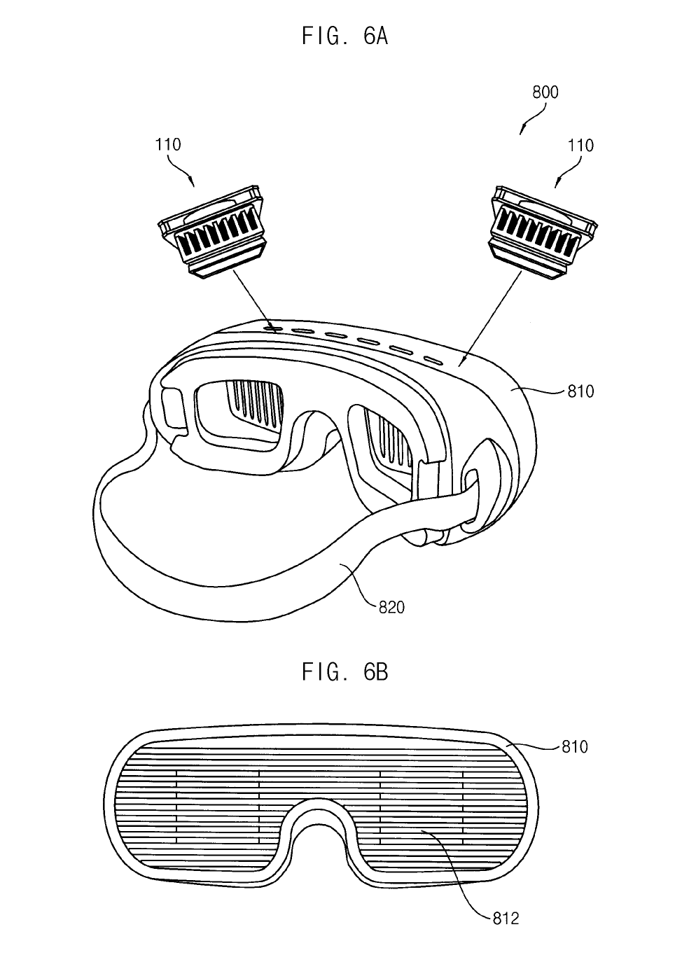

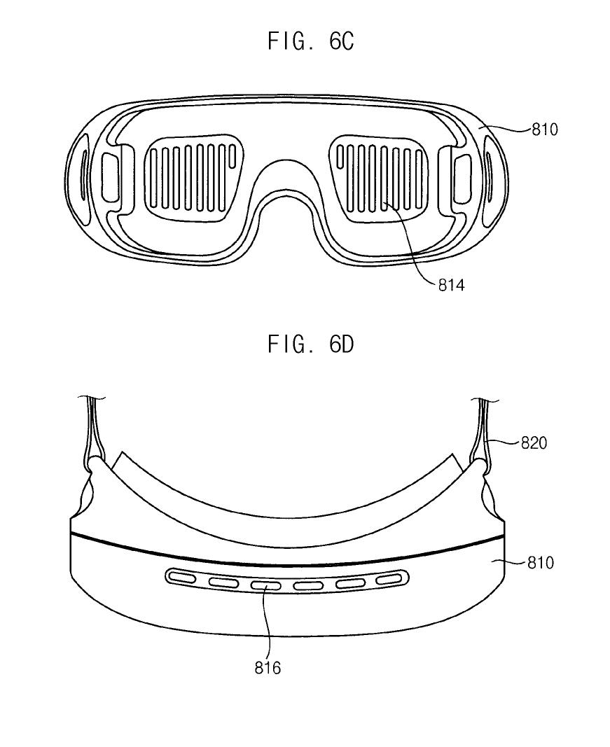

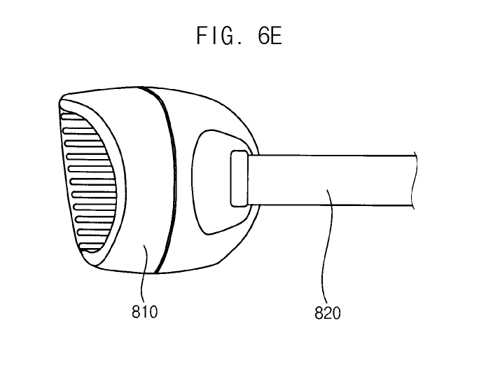

FIG. 6A is a perspective view illustrating an eyeball cooling goggle according to example embodiments, FIG. 6B is a front view illustrating an eyeball cooling goggle of FIG. 6A, FIG. 6C is a rear view illustrating an eyeball cooling goggle of FIG. 6A, FIG. 6D is a plain view illustrating an eyeball cooling goggle of FIG. 6A, and FIG. 6E is a side view illustrating an eyeball cooling goggle of FIG. 6A.

Referring to FIGS. 6A through 6E, an eyeball cooling goggle 800 includes the eyeball cooling module 110, an acceptance unit 810 and a flexible band 820.

The eyeball cooling module 110 generates a cooling stimulation to eyeballs, is explained with reference to FIGS. 1 and 2, and thus, detailed description of the eyeball cooling module 110 will be omitted.

The acceptance unit 810 is opposed to a portion opposed to eyeballs of a human that wears the eyeball cooling goggle 800 and accepts the eyeball cooling module 110. The eyeball cooling module 110 is accepted in the acceptance unit 810 such that the heat absorption unit is opposed to the eyes of the human.

The acceptance unit 810 includes heat absorption holes 814, first heat dissipation holes 812 and second heat dissipation holes 816.

The heat absorption holes 814 are formed on a first surface apposed to the human eyeballs and transfer heat from the eyeballs to the eyeball cooling module 110. The first surface may be a rear surface of the eyeball cooling goggle 800. The first heat dissipation holes 812 are formed on a second surface opposed to the first surface and dissipate the heat from the eyeball cooling module 110. The second surface may be a front surface of the eyeball cooling goggle 800. The second heat dissipation holes 816 are formed on a third surface perpendicular to the first surface and the second surface and dissipate the heat from the eyeball cooling module 110.

At least one of the heat absorption holes 814, the first heat dissipation holes 812 and the second heat dissipation holes 816 may be applicable to the eyeball cooling eyepatch 600 and the eyeball cooling eyeglasses 700.

The flexible band 820 is coupled from both ends of the acceptance unit 810 to form a closed loop and prevents the eyeball cooling goggle 800 from rolling down.

Accordingly, the eyeball fever may be quickly and easily relieved by wearing the eyeball cooling goggle 800 that generates the cooling stimulation to the eyeballs of the human.

The foregoing is illustrative of example embodiments and is not to be construed as limiting thereof. Although a few example embodiments have been described, those skilled in the art will readily appreciate that many modifications are possible in the example embodiments without materially departing from the novel teachings and advantages of the present inventive concept. Accordingly, all such modifications are intended to be included within the scope of the present inventive concept as defined in the claims. Therefore, it is to be understood that the foregoing is illustrative of various example embodiments and is not to be construed as limited to the specific example embodiments disclosed, and that modifications to the disclosed example embodiments, as well as other example embodiments, are intended to be included within the scope of the appended claims.

### ABILITY OF INDUSTRIAL UTILITY

The eyeball cooling module, the eyeball cooling apparatus, the eyeball cooling eyepatch, the eyeball cooling eyeglasses and the eyeball cooling goggle according to example embodiments may generate cooling stimulation to eyeballs of a human. Therefore, the example embodiments may be used for relieving fatigue of eyes and eyeballs at home and may be used for reducing eyeball fever after operation on eyes and eyeballs at hospitals.

What is claimed is:

1. An eyeball cooling module comprising:
a cooling device to generate a cooling stimulation to human eyeballs by absorbing heat from a first side in which the human eyeballs are disposed and dissipating heat to a second side opposed to the first side; and
a heat dissipation unit, disposed at the second side of the cooling device, to dissipate heat from the second side of the cooling device externally.

2. The eyeball cooling module of claim 1, wherein the cooling device includes a Peltier device that includes two metal plates spaced apart from each other, and the Peltier device absorbs heat from the first side and dissipates heat to the second side when a potential occurs between the two metal plates.

3. The eyeball cooling module of claim 2, wherein the Peltier device comprises:
a heat absorption unit including a first metal plate and a second metal plate that are disposed at the first side and are spaced apart from each other, the potential being induced between the first metal plate and the second metal plate;
a heat dissipation unit including a third metal plate disposed at the second side;
a p-type device disposed between the first metal plate and the third metal plate; and
an n-type device disposed between the second metal plate and the third metal plate.

4. The eyeball cooling module of claim 1, wherein the heat dissipation unit comprises a heat dissipation pad, a heat dissipation plate and a heat dissipation pan which are disposed in consecutive order from the second side of the cooling device.

5. An eyeball cooling apparatus comprising:
an eyeball cooling module comprising:
a cooling device to generate a cooling stimulation to human eyeballs by absorbing heat from a first side in which the human eyeballs are disposed and dissipating heat to a second side opposed to the first side; and
a heat dissipation unit, disposed at the second side of the cooling device, to dissipate heat from the second side of the cooling device externally;
a power supply unit to provide a power supply voltage to the eyeball cooling module; and
a cooling stimulation control unit to output a cooling stimulation control signal that controls the cooling stimulation to the power supply unit.

6. The eyeball cooling apparatus of claim 5, wherein the power supply unit is configured to control a level of the power supply voltage to control a strength of the cooling stimulation, in response to the cooling stimulation control signal.

7. The eyeball cooling apparatus of claim 5, wherein the power supply unit is configured to control a supply time of the power supply voltage to control a time of the cooling stimulation, in response to the cooling stimulation control signal.

8. The eyeball cooling apparatus of claim 5, wherein the power supply unit is configured to control a supply pattern of the power supply voltage to control a pattern of the cooling stimulation, in response to the cooling stimulation control signal.

9. The eyeball cooling apparatus of claim 5, wherein the cooling device includes a Peltier device that includes two metal plates spaced apart from each other, and the Peltier device absorbs heat from the first side and dissipates heat to the second side when a potential occurs between the two metal plates.

10. The eyeball cooling apparatus of claim 5, wherein the heat dissipation unit comprises a heat dissipation pad, a heat dissipation plate and a heat dissipation pan which are disposed in consecutive order from the second side of the cooling device.

11. An eyeball cooling eyepatch, comprising:
an eyeball cooling module comprising:
a cooling device to generate a cooling stimulation to human eyeballs by absorbing heat from a first side in which the human eyeballs are disposed and dissipating heat to a second side opposed to the first side; and
a heat dissipation unit, disposed at the second side of the cooling device, to dissipate heat from the second side of the cooling device externally;
an acceptance unit, opposed to the human eyeballs, to accept the eyeball cooling module; and
a flexible band coupled from both ends of the acceptance unit.

12. An eyeball cooling eyeglasses, comprising:
an eyeball cooling module comprising:
a cooling device to generate a cooling stimulation to human eyeballs by absorbing heat from a first side in which the human eyeballs are disposed and dissipating heat to a second side opposed to the first side; and
a heat dissipation unit, disposed at the second side of the cooling device, to dissipate heat from the second side of the cooling device externally;
an acceptance unit, opposed to the human eyeballs, to accept the eyeball cooling module; and
ear-holding units that extend from both ends of the acceptance unit and have configurations to attach from human ears.

13. An eyeball cooling goggle comprising:
an eyeball cooling module comprising:
a cooling device to generate a cooling stimulation to human eyeballs by absorbing heat from a first side in which the human eyeballs are disposed and dissipating heat to a second side opposed to the first side; and
a heat dissipation unit, disposed at the second side of the cooling device, to dissipate heat from the second side of the cooling device externally;
an acceptance unit, opposed to the human eyeballs, to accept the eyeball cooling module, the acceptance unit including heat absorption holes formed on a first surface opposed to the human eyeballs; and
a flexible band coupled from both ends of the acceptance unit.

14. The eyeball cooling goggle of claim 13, wherein the acceptance unit further comprises first heat dissipation holes formed on a second surface opposed to the first surface.

15. The eyeball cooling goggle of claim 14, wherein the acceptance unit further comprises second heat dissipation holes formed on a third surface perpendicular to the first surface and the second surface.
